(19) United States
(12) Patent Application Publication
(10) Pub. No.: US 2014/0356862 A1
(43) Pub. Date: Dec. 4, 2014

(54) SYSTEM FOR ON-LINE MONITORING AND CONTROLLING OF CHEMICAL REACTIONS IN REACTORS

(71) Applicant: ASPECT AI LTD., Netanya (IL)

(72) Inventor: Uri Rapoport, Moshav Ben Shemen (IL)

(21) Appl. No.: 14/345,504

(22) PCT Filed: Sep. 19, 2012
(86) PCT No.: PCT/IL2012/000341

§ 371 Date: Mar. 18, 2014

Related U.S. Application Data
(60) Provisional application No. 61/537,108, filed on Sep. 21, 2011.

(51) Int. Cl. C12Q 1/02; G01N 33/50

(52) U.S. Cl. CPC C12Q 1/02; G01N 33/5005
USPC 435/5; 435/287.1; 435/29

(57) ABSTRACT

The application describes an MRD-based reactor. The reactor is characterized by a continuous wall portion, and is in connection with a MRD, adapted for performing localized NMR spectroscopy of the medium inside the reactor. MRD-based reactors, in which the MRD is at least partially inside the reactor or reaction media, and those in which the MRD accommodates the reactor, are also introduced. Lastly, the invention teaches an in situ method for controlling and analyzing of a reaction. The method makes use of an MRD-based reactor; and comprises applying a magnetic field within the reactor, especially for performing a plurality of localized spectroscopic measurements and either real time or offline analyzing and/or controlling of reactions in the flowing media.

### FIELD OF THE INVENTION

The present invention generally pertains to a system and method for ensuring that time resolved reactions are identical in a sample and in the medium which provided the sample.

### BACKGROUND OF THE INVENTION

Chemical reactions, especially those involving biological material, are usually not first order. Especially for production of pharmaceutical products, the environment in the vessel must be closely controlled to enable the proper expression of biochemical reactions for the production of the desired products.

Present techniques rely on removing samples of the reaction medium from the reaction vessel and analyzing the samples in a separate, often stand-alone analysis device. No matter how carefully the samples are removed from the reaction vessel, and no matter what care is taken to preserve the correct operating conditions during transport from the reaction vessel to the analysis device, time will necessarily have passed before the analysis can be made.

This passage of time has several deleterious effects. The first and more obvious one is that control of the reaction is thereby rendered more difficult, if not impossible, since the results of the analysis can not be used before they are generated. For example, take an analysis result that shows that a change needs to be made within a minute of the time the sample was taken from the reaction vessel. If the time between taking the sample from the reaction vessel and receiving the results of the analysis is greater than a minute, by the time it is known that the change needs to be made, it will be too late to make it.

Another deleterious effect is most prominent in biochemical processes, although it occurs in other processes as well. It is the effect of aging. For example, cells do not stop either growing or aging if they are removed from a reaction vessel. A cell in its lag phase may well have multiplied into many cells between sample taking and analysis, it may have entered its log phase, and it may even have gone into decline. Therefore, the sample that is analyzed is not the same as the sample that was taken, and is therefore not only not the same as the reaction medium it purports to represent, but may differ significantly from that reaction medium. Therefore, using the results of analysis of a sample to control a process may well lead to undesirable results. This is a well-known problem in the industry and much effort has gone into minimizing changes in the sample between time of taking and time of analysis.

Another result of the changes in the sample between taking and analysis is that it is difficult to use samples to calculate the total amount of product created during a process. Analysis of the samples for quantity of product may be made and the area under the quantity-vs-time curve, the integral of the curve, will give a total amount of product. However, even small differences between measured amount of product and actual amount of product may add up to a significant difference between measured output and actual output. Therefore, product quantity must often be found from a separate analysis of the reaction medium after removal from the vessel.

U.S. Pat. No. 6,103,934 to Hallinan et al. discloses a process control method for producing acetic acid by catalyzed carbonylation of methanol in which various reactor component concentrations, e.g., active catalyst, methyl iodide, water and methyl acetate are measured using an infrared analyzer. The concentrations are adjusted in response to the measurements taken to optimize the acetic acid reaction. However, this patent teaches an IR analyzer downstream of the reactor and also teaches a process which does not change with time.

U.S. Pat. No. 6,228,650 to Moore et al. discloses controlling concentration of alkylation catalyst components Hydrofloric acid, acid soluble oil (ASO) and water, by measuring a continuously flowing catalyst slipstream in an IR analyzer and using the results to vary the temperature of stripping fluid in order to control ASO levels within a preferred range. However, this patent teaches a continuously flowing catalyst slipstream piped to an analyzer/controller which is separate from the reactor and also teaches a process which does not change with time.

U.S. Pat. No. 5,862,060 to Murray, Jr. discloses controlling chemical processes using compositional data, as the basis for control using NIR (Near InfraRed) spectroscopy which allows for on-line measurements in real time. A calibration set of NIR spectra binding the acceptable process space for a particular controlled property is assembled and a multi-variant statistical method is applied to the calibration step to identify a small number (2-4) of the characteristics of the set governing the controlled property. Thus a complex process can be controlled in such a way as to provide a substantially invariant product composition. However, this patent teaches NIR spectroscopy downstream of the reactor and also teaches a process which does not change with time.

Patent US2009/0197294 discloses an MRD-based reactor for use as a fermentor. Patent US2009/0197294 does not teach time-resolved analysis of the sample.

It is therefore a long-felt need for a means of analyzing samples of a reaction medium so that no properties of the sample will have changed between the time the sample is taken and the time it is analyzed and where the sample is not removed from the main body of the reaction medium.

### SUMMARY OF THE INVENTION

It is an object of the present invention to disclose a reactor arrangement for continuous and synchronized MRD analysis of a time resolved reaction, where the reactor arrangement is characterized by a reaction vessel; a flowable reaction medium (FRM) provided within the vessel, the FRM is further characterized by at least one time resolved reaction (TRR) occurring during a first time period (1^{st} TP); and an MRD at least temporarily accommodating in its volume of interest (VOI) at least a portion of the FRM (the sample, SRM), the SRM is further characterized by the TRR and may occur during a given second time period (2^{nd} TP); where the SRM is continuously and effectively homogeneous with the FRM, the 1^{st} TP and 2^{nd} TP occur simultaneously, in a manner that the MRD analysis of the SRM is identical to and simultaneous with an MRD analysis of the FRM.

It is in the scope of the invention wherein the MRD is configured such that it is capable of time resolved analysis of the SRM within its VOI.

It is in the scope of the invention wherein the MRD is further configured such that it is capable spatially-resolved analysis of the SRM within its VOI.

It is in the scope of the invention wherein the reactor arrangement additionally comprises an alarm capable of responding to at least one component of the MRD analysis within at least one range.

It is in the scope of the invention wherein the reactor arrangement is adapted for detecting biologically active material.

It is in the scope of the invention wherein the MRD is adapted for detecting ATP or ADP.

It is in the scope of the invention wherein the biologically active material is detected via a phosphorous resonance.

It is in the scope of the invention wherein the biologically active material is a contaminant of the reaction medium.

It is in the scope of the invention wherein the reactor arrangement is adapted for fermentation.

Another object of the invention is to disclose a method of continuous and synchronous MRD analysis of a time resolved reaction. The method comprises steps of obtaining a reaction vessel; providing a flowable reaction medium (FRM) within the reaction vessel, the FRM is further characterized by at least one time resolved reaction (TRR) occurring during a first time period (1^{st} TP); and at least temporarily accommodating in the volume of interest (VOI) of an MRD at least a portion of the FRM (the sample, SRM), the SRM is further characterized by the TRR and may occur during a given second time period (2^{nd} TP); where the SRM is continuously and effectively homogeneous with the FRM; the 1^{st} TP and 2^{nd} TP occur simultaneously, in a manner that the MRD analysis of the SRM is identical to and simultaneous with an MRD analysis of the FRM.

It is in the scope of the invention wherein the MRD is configured such that it is capable of time resolved analysis of said SRM within its VOI.

It is in the scope of the invention wherein the MRD is further configured such that it is capable spatially-resolved analysis of said SRM within its VOI.

It is in the scope of the invention wherein the method additionally comprises a step of controlling at least one operating condition of said reaction vessel via a feedback control system responsive to said MRD analysis.

It is in the scope of the invention wherein the method additionally comprises a step of activating an alarm in response to at least one component of said MRD analysis within at least one range.

It is in the scope of the invention wherein the method is adapted for detecting biologically active material.

It is in the scope of the invention wherein the method is adapted for an MRD adapted for detecting ATP or ADP.

It is in the scope of the invention wherein the method is adapted for detecting a biologically active material via a phosphorous resonance.

It is in the scope of the invention wherein the method is adapted for a reaction vessel adapted for fermentation.

### BRIEF DESCRIPTION OF THE FIGURES

In order to better understand the invention and its implementation in practice, a plurality of embodiments will now be described, by way of non-limiting example only, with reference to the accompanying drawings, wherein

FIG. 1 schematically illustrates changes in a physical property of a reaction medium according to prior art;

FIG. 2 schematically illustrates changes in a physical property of a reaction medium according to the present invention;

FIG. 3 schematically illustrates an embodiment of the method of operation;

FIG. 4 schematically illustrates an embodiment of the method of operation where operating conditions are altered based on the analysis of the sample; and

FIG. 5 schematically illustrates an embodiment of the method of operation where an alarm is activated based on the analysis of the sample.

### DETAILED DESCRIPTION OF THE PREFERRED EMBODIMENTS

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a means and method for reactors with MRI-based monitoring, analysis or control.

None of the prior art references disclose a reactor that utilizes MRD spectroscopy for analyzing and controlling on-line and in situ a reaction provided in a reactor in real-time. None of the literature cited teaches the adaptation of such reactor for ensuring that the measured properties of the sample and the properties of the reaction medium will be identical and that the sample will at all times remain an integral part of the reaction medium.

The term ‘magnetic resonance detector’ (MRD) applies hereinafter to any Magnetic Resonance Imaging (MRI) device, any Nuclear Magnetic resonance (NMR) spectroscope, any Electron Spin Resonance (ESR) spectroscope, any Nuclear Quadrupole Resonance (NQR) or a combination thereof.

The term ‘flowable reaction medium’ applies hereinafter to any flowable matter, before, after or in the process of a reaction. The medium is selected in a non-limiting manner from a group consisting of gas, liquid, flowing solids such as particles, especially nanoparticles and micronic particles, sols, gels, sol-gels, colloids, emulsions, suspensions, dispersions, liposomes, aggregates, crystals, cells including red cells and stem cells, seeds, or a combination thereof.

The term ‘Reactor’ applies hereinafter to chemical, biological and/or physical reactors or bioreactors, namely to vessels that are designed for chemical, biological and/or physical reaction to occur inside of them. The reactor is normally but not exclusively characterized as a tank reactor—a tank that is usually enclosed to keep contaminants out of the reaction vessel, or envelope, tubular reactor—a pipe or tube or a combination thereof. Both types can be used according to the present invention as continuous reactors or batch reactors. The reactor may run at steady-state, but can also be operated in a transient state. The reactor may accommodate one or more solids (reagents, catalyst, or inert materials), but the reagents and products are typically liquids and gases. Preferably, yet not exclusively, the medium is liquid.

The term ‘time resolved reaction’ (TRR) applies hereinafter to any reaction which varies over time. The reaction is selected in a non-limiting manner from a group consisting of inorganic reactions, organic reactions, cell-free biological reactions, biological reactions of living cells, or biological reactions utilizing dead cells, or a combination thereof.

The term ‘operating condition’ applies hereinafter to any physical parameter of the flowable medium which may be monitored or controlled by the reactor. Operating conditions are selected in a non-limiting manner from a group consisting of temperature, pressure, pH, concentration of at least one reactant, mixer speed, impeller speed, or rotation rate of said chamber, or any combination thereof.

The term ‘biologically active material’ applies hereinafter to any material where the reactions are biological reactions. Biological material is typically, but not exclusively, selected from a group consisting of cells including red cells and stem cells, bacteria, yeasts, algae, viruses, or tissues, or any combination thereof.

The term ‘plurality’ applies hereinafter to any integer greater than or equal to one.

The term ‘about’ refers hereinafter to a tolerance of ±20% of the defined measurement.

According to one embodiment of the present invention, the desired product is a pharmaceutical.

According to another embodiment of the invention, the desired product is a chemical such as, but not limited to, alcohol or acetic acid.

According to another embodiment of the present invention, biological contamination of liquids may be determined from the quantity of ATP and/or ADP in the liquid, since ATP especially is a reliable marker of biological contamination. In the preferred embodiment, quantities of ATP and/or ADP are determined from the phosphorous (P₃₁) resonance, although other resonances may be used. Liquids of interest for determination of biological contamination are selected in a non-limiting manner from a group consisting of wastewater, sewage, potable water, milk, fruit, fruit juice, vegetables, vegetable juice, juice drinks, flavored water, sparkling water, wine, beer, whisky, liqueur, brandy, tea, coffee, fruit tea, herb tea, sugar, glucose, fructose, sucrose, artificial sweetener, any mixture thereof with water, or any combination thereof.

According to another embodiment of the present invention, ATP content is used to determine the fraction of cells in fermentors. Testing for ATP in this way allows total ATP content to be determined without the need to lyse cells which may be present in the media.

According to another embodiment of the present invention, the desired reaction is a fermentation process and desired outcome is an alcoholic drink. In this embodiment, the reaction medium is typically, but not exclusively, selected in a non-limiting manner from a group consisting of fruit, fruit juice, vegetables, vegetable juice, malted or unmalted grain, milk, honey, any mixture thereof with water, or any combination thereof.

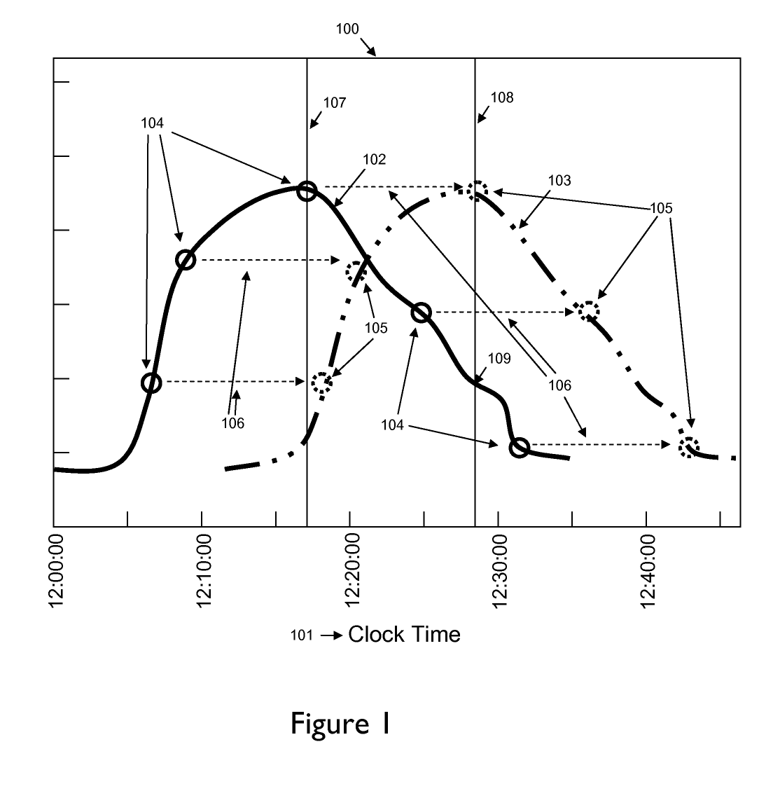

Reference is made now to FIG. 1, schematically depicting prior art graphically. In the graph (100) a property of a biological reaction is shown as a function of clock time (101). The reaction (102) starts with an inoculum at 12:00 and is complete by 12:35. At various times during the reaction (102), samples are taken (104). Said samples are removed from the reactor and, with proper precautions to ensure that proper conditions are preserved, are analyzed (104), producing a plot of the property vs. time (103). The time taken to transport the sample to the analyzer is, in this example, about 11 minutes (106). The property of the sample which is of interest has changed in the interval, so it is different at the time of analysis than it was at the time the sample was taken; the plot of the property of the reaction vs time (102) is different from the plot of the property as measured from the samples vs. time (103).

Furthermore, If changes need to be made to the reactor operating conditions—if, for example, material needs to be withdrawn from the reactor and fresh material needs to be added before the reaction declines (107), by the time the sample is analyzed (108), the reaction may be already far into its decline phase (109).

Another example of a disadvantage of prior art may be derived from FIG. 1. One property of interest is quantity of product created in a reaction. If the solid curve (102) in FIG. 1 shows quantity of product in the reaction medium vs. time, then the area under the solid curve gives the total amount of product created by the process. The dashed curve (103) is then quantity of product in the reaction medium as measured from the samples and the area under the dashed curve gives the measured total amount of product in the samples. Since the shapes of the curves differ, the measured amount of product differs from the actual amount of product. Since the shape of the solid curve (102) is not known, the difference between the actual amount of product and the measured amount is not known. In addition, the difference between measured total product and actual total produce may also change between batches in a batch reactor or over time for a continuous reactor.

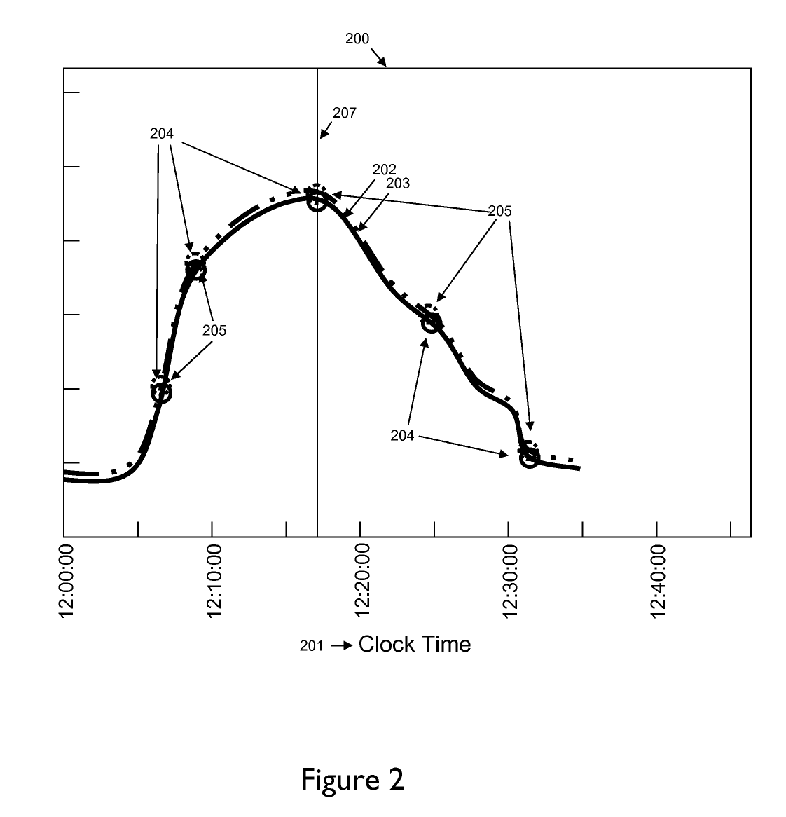

Reference is made now to FIG. 2, schematically depicting the present invention. In the graph (200) the same property of the same biological reaction as FIG. 1 is shown as a function of clock time (201). The reaction (202) starts with an inoculum at 12:00 and is complete by 12:35. In the present invention, samples are not removed from the reactor; analysis is done in-situ. The sample remains part of the material in the reactor at all times. Sampling times (204) and analysis times (205) are identical. The plot of sample property vs. time (203) is necessarily identical to the plot of property vs time (202). The two curves are shown in FIG. 2 slightly displaced for clarity.

If changes need to be made to the reactor operating conditions—if, for example, material needs to be withdrawn from the reactor and fresh material needs to be added before the reaction declines (207), this can be done in a timely manner (207).

FIG. 2 shows an advantage of the present invention over prior art. One property of interest is quantity of product created in a reaction. If the solid curve (202) in FIG. 1 shows quantity of product in the reaction medium vs. time, then the area under the solid curve gives the total amount of product created by the process. The dashed curve (203) is then quantity of product in the reaction medium as measured from the samples and the area under the dashed curve gives the measured total amount of product in the samples. Since the shapes of the solid curve (202) and the dashed curve (203) are necessarily the identical, the areas under them are necessarily the same and the measured quantity of product is necessarily the same as the actual total product, so that further analysis of the product may be unnecessary.

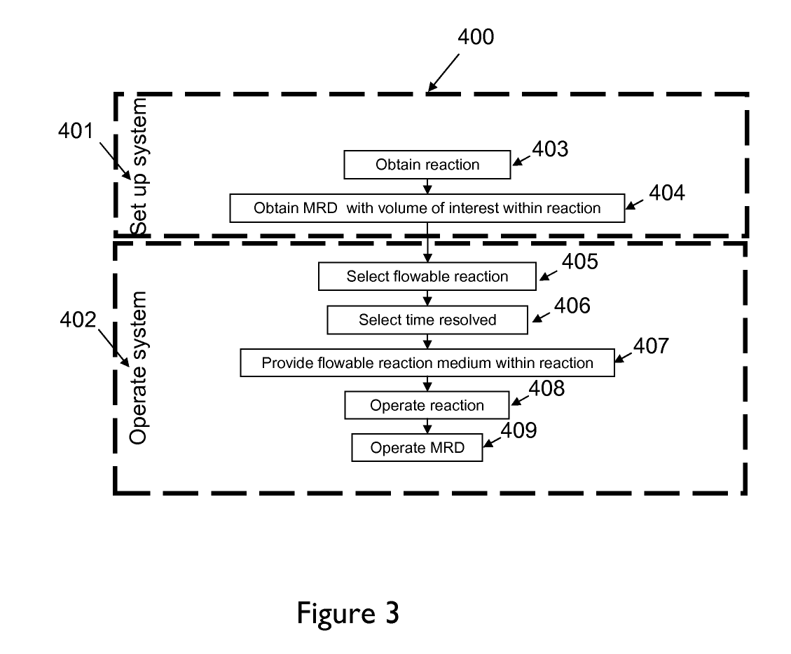

Reference is made now to FIG. 3, which schematically depicts the method of operation of another embodiment of the invention (300). Set-up of the system (301) is done once; operating the system (302) may be done many times. During set-up (301), a reactor vessel is obtained (302) with an MRD with a volume of interest within the reactor vessel (303). During operation, a flowable reaction medium (304) and a time resolved reaction to analyze (305) are selected. The flowable reaction medium is then provided in the interior of the reaction vessel (306) and the vessel is operated (307). During operation, samples of the reaction medium are analyzed by the MRD (308). The results of said analysis may be monitored or stored.

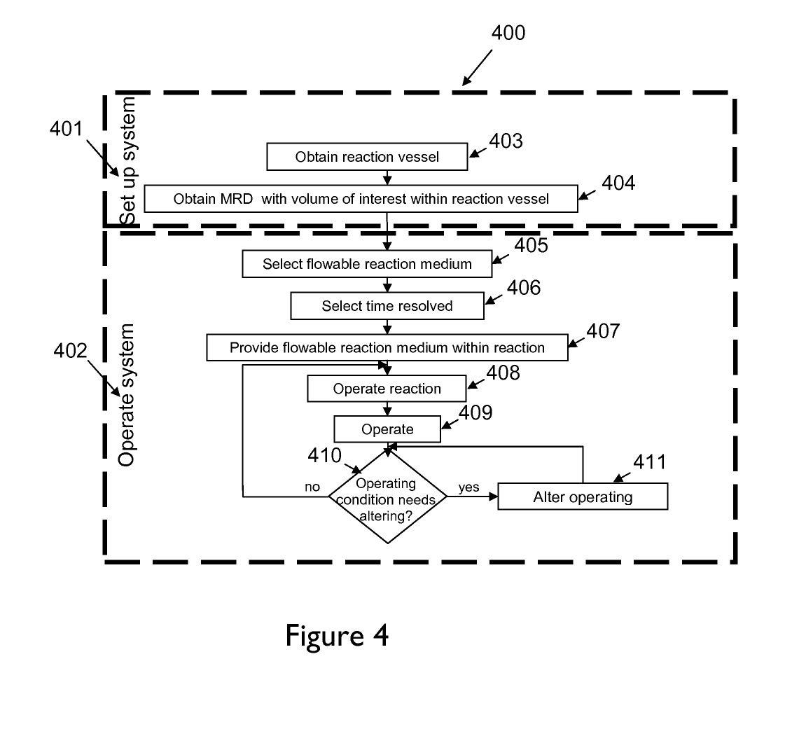

Reference is made now to FIG. 4, which schematically depicts the method of operation of an embodiment of the invention (400). Set-up of the system (401) is done once; operating the system (402) may be done many times. During set-up (401), a reactor vessel is obtained (403) with an MRD with a volume of interest within the reactor vessel (404). During operation, a flowable reaction medium (405) and a time resolved reaction to analyze (406) are selected. The flowable reaction medium is then provided in the interior of the reaction vessel (407) and the vessel is operated (408). During operation, samples of the reaction medium are analyzed by the MRD (409). The results of said analysis are monitored and may be stored. Based on the results of the analysis, if an operating condition needs to be altered (410), it may be altered (411) to ensure that the reaction continues under the proper conditions (408). In other words, the system is a feedback system.

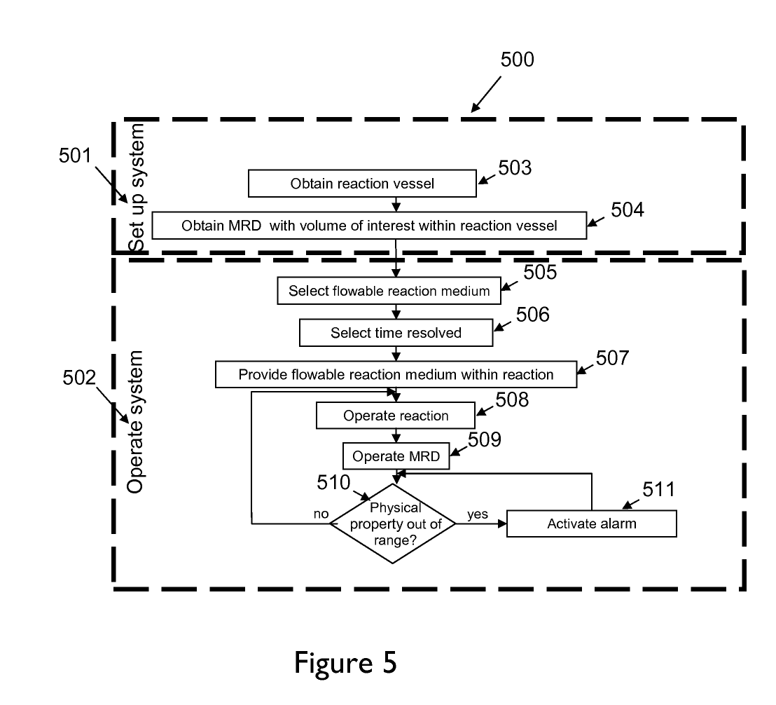

Reference is made now to FIG. 5, which schematically depicts the method of operation of another embodiment of the invention (500). Set-up of the system (501) is done once; operating the system (502) may be done many times. During set-up (501), a reactor vessel is obtained (503) with an MRD with a volume of interest within the reactor vessel (503). During operation, a flowable reaction medium (505) and a time resolved reaction to analyze (506) are selected. The flowable reaction medium is then provided in the interior of the reaction vessel (507) and the vessel is operated (508). During operation, samples of the reaction medium are analyzed by the MRD (509). The results of said analysis are monitored and may be stored. Based on the results of the analysis, if an operating condition needs to be altered (510), it may be altered (511) to ensure that the reaction continues under the proper conditions (508).

In another embodiment of the method of operation, the reactor may be shut down safely if an alarm condition is detected.

In another embodiment of the method of operation, any combination of the above may be part of the method of operation.

1-23. (canceled)

24. A reactor arrangement for continuous and synchronized MRD analysis of a time resolved reaction, said arrangement is characterized by:
a. a reaction vessel;
b. a flowable reaction medium (FRM) provided within said vessel, said FRM is further characterized by at least one time resolved reaction (TRR) occurring during a first time period (1^{st} TP); and
c. an MRD at least temporarily accommodating in its volume of interest (VOI) at least a portion of said FRM (the sample, SRM), said SRM is further characterized by said TRR and may occur during a given second time period (2^{nd} TP);
said SRM is continuously and effectively homogeneous with said FRM; said 1^{st} TP and 2^{nd} TP occur simultaneously, in a manner that said MRD analysis of said SRM is identical to and simultaneous with an MRD analysis of said FRM.

25. The reactor arrangement of claim 24, wherein measured integrated quantities determined from said MRD analysis are necessarily equal to actual integrated quantities.

26. The reactor arrangement of claim 24, wherein said MRD is configured such that it is capable of time resolved analysis of said SRM within its VOI.

27. The reactor arrangement of claim 26, wherein said MRD is further configured such that it is capable spatially-resolved analysis of said SRM within its VOI.

28. The reactor arrangement of claim 24, wherein at least one of the following is true:
a. Said reactor arrangement additionally comprises an alarm capable of responding to at least one component of said MRD analysis within at least one range;
b. said reactor arrangement additionally comprises a shutdown procedure capable of responding to at least one component of said MRD analysis within at least one range;
and any combination thereof.

29. The reactor arrangement of claim 24, adapted for detecting biologically active material.

30. The reactor arrangement of claim 29, wherein at least one of the following is true:
a. said MRD is adapted for detecting ATP or ADP;
b. said biologically active material is detected via a phosphorous resonance;
c. said biologically active material is a contaminant of said reaction medium;
d. said reactor arrangement is adapted for fermentation;
and any combination thereof.

31. The reactor arrangement of claim 24, wherein said reactor arrangement is adapted for fermentation.

32. A method of continuous and synchronous MRD analysis of a time resolved reaction, comprising steps of
a. obtaining a reaction vessel;
b. providing a flowable reaction medium (FRM) within said reaction vessel, said FRM is further characterized by at least one time resolved reaction (TRR) occurring during a first time period (1^{st} TP); and
c. at least temporarily accommodating in the volume of interest (VOI) of an MRD at least a portion of said FRM (the sample, SRM), said SRM is further characterized by said TRR and may occur during a given second time period (2^{nd} TP);
said SRM is continuously and effectively homogeneous with said FRM; said 1^{st} TP and 2^{nd} TP occur simultaneously, in a manner that said MRD analysis of said SRM is identical to and simultaneous with an MRD analysis of said FRM.

33. The method of claim 32, wherein measured integrated quantities determined from said MRD analysis are necessarily equal to actual integrated quantities.

34. The method of claim 32, wherein said MRD is configured such that it is capable of time resolved analysis of said SRM within its VOI.

35. The method of claim 34, wherein said MRD is further configured such that it is capable spatially-resolved analysis of said SRM within its VOI.

36. The method of claim 32, additionally comprising at least one of the following steps:
a. a step of controlling at least one operating condition of said reaction vessel via a feedback control system responsive to said MRD analysis;
b. a step of activating an alarm in response to at least one component of said MRD analysis within at least one range;
c. a step of providing a shutdown procedure capable of responding to at least one component of said MRD analysis within at least one range;
d. and any combination thereof.

37. The method of claim 32, adapted for detecting biologically active material.

38. The method of claim 37, additionally comprising at least one of the following steps:
a. adapting said MRD for detecting ATP or ADP;
b. detecting said biologically active material via a phosphorous resonance.
c. detecting said biologically active material as a contaminant of said reaction medium;
d. adapting said reaction vessel for fermentation;
e. and any combination thereof.

39. The method of claim 32, additionally comprising a step of adapting said reaction vessel for fermentation.
